SEQUENCE LISTING (20 sequences)

SEQ ID NO: 1 - DNA, 2710 bp, Homo sapiens
atgagacagactttgccttgtatctacttttgggggggccttttgccctttgggatgctgtgtgcatcctccaccaccaagtgcactgttagccatgaagttgctgactgcagccacctgaagttgactcaggtacccgatgatctacccacaaacataacagtgttgaaccttacccataatcaactcagaagattaccagccgccaacttcacaaggtatagccagctaactagcttggatgtaggatttaacaccatctcaaaactggagccagaattgtgccagaaacttcccatgttaaaagttttgaacctccagcacaatgagctatctcaactttctgataaaacctttgccttctgcacgaatttgactgaactccatctcatgtccaactcaatccagaaaattaaaaataatccctttgtcaagcagaagaatttaatcacattagatctgtctcataatggcttgtcatctacaaaattaggaactcaggttcagctggaaaatctccaagagcttctattatcaaacaataaaattcaagcgctaaaaagtgaagaactggatatctttgccaattcatctttaaaaaaattagagttgtcatcgaatcaaattaaagagttttctccagggtgttttcacgcaattggaagattatttggcctctttctgaacaatgtccagctgggtcccagccttacagagaagctatgtttggaattagcaaacacaagcattcggaatctgtctctgagtaacagccagctgtccaccaccagcaatacaactttcttgggactaaagtggacaaatctcactatgctcgatctttcctacaacaacttaaatgtggttggtaacgattcctttgcttggcttccacaactagaatatttcttcctagagtataataatatacagcatttgttttctcactctttgcacgggcttttcaatgtgaggtacctgaatttgaaacggtcttttactaaacaaagtatttcccttgcctcactccccaagattgatgatttttcttttcagtggctaaaatgtttggagcaccttaacatggaagataatgatattccaggcataaaaagcaatatgttcacaggattgataaacctgaaatacttaagtctatccaactcctttacaagtttgcgaactttgacaaatgaaacatttgtatcacttgctcattctcccttacacatactcaacctaaccaagaataaaatctcaaaaatagagagtgatgctttctcttggttgggccacctagaagtacttgacctgggccttaatgaaattgggcaagaactcacaggccaggaatggagaggtctagaaaatattttcgaaatctatctttcctacaacaagtacctgcagctgactaggaactcctttgccttggtcccaagccttcaacgactgatgctccgaagggtggcccttaaaaatgtggatagctctccttcaccattccagcctcttcgtaacttgaccattctggatctaagcaacaacaacatagccaacataaatgatgacatgttggagggtcttgagaaactagaaattctcgatttgcagcataacaacttagcacggctctggaaacacgcaaaccctggtggtcccatttatttcctaaagggtctgtctcacctccacatccttaacttggagtccaacggctttgacgagatcccagttgaggtcttcaaggatttatttgaactaaagatcatcgatttaggattgaataatttaaacacacttccagcatctgtctttaataatcaggtgtctctaaagtcattgaaccttcagaagaatctcataacatccgttgagaagaaggttttcgggccagctttcaggaacctgactgagttagatatgcgctttaatccctttgattgcacgtgtgaaagtattgcctggtttgttaattggattaacgagacccataccaacatccctgagctgtcaagccactacctttgcaacactccacctcactatcatgggttcccagtgagactttttgatacatcatcttgcaaagacagtgccccctttgaactctttttcatgatcaataccagtatcctgttgatttttatctttattgtacttctcatccactttgagggctggaggatatctttttattggaatgtttcagtacatcgagttcttggtttcaaagaaatagacagacagacagaacagtttgaatatgcagcatatataattcatgcctataaagataaggattgggtctgggaacatttctcttcaatggaaaaggaagaccaatctctcaaattttgtctggaagaaagggactttgaggcgggtgtttttgaactagaagcaattgttaacagcatcaaaagaagcagaaaaattatttttgttataacacaccatctattaaaagacccattatgcaaaagattcaaggtacatcatgcagttcaacaagctattgaacaaaatctggattccattatattggttttccttgaggagattccagattataaactgaaccatgcactctgtttgcgaagaggaatgtttaaatctcactgcatcttgaactggccagttcagaaagaacggataggtgcctttcgtcataaattgcaagtagcacttggatccaaaaactctgtac

SEQ ID NO: 2 - protein, 904 aa, Homo sapiens
MRQTLPCIYFWGGLLPFGMLCASSTTKCTVSHEVADCSHLKLTQVPDDLPTNITVLNLTHNQLRRLPAANFTRYSQLTSLDVGFNTISKLEPELCQKLPMLKVLNLQHNELSQLSDKTFAFCTNLTELHLMSNSIQKIKNNPFVKQKNLITLDLSHNGLSSTKLGTQVQLENLQELLLSNNKIQALKSEELDIFANSSLKKLELSSNQIKEFSPGCFHAIGRLFGLFLNNVQLGPSLTEKLCLELANTSIRNLSLSNSQLSTTSNTTFLGLKWTNLTMLDLSYNNLNVVGNDSFAWLPQLEYFFLEYNNIQHLFSHSLHGLFNVRYLNLKRSFTKQSISLASLPKIDDFSFQWLKCLEHLNMEDNDIPGIKSNMFTGLINLKYLSLSNSFTSLRTLTNETFVSLAHSPLHILNLTKNKISKIESDAFSWLGHLEVLDLGLNEIGQELTGQEWRGLENIFEIYLSYNKYLQLTRNSFALVPSLQRLMLRRVALKNVDSSPSPFQPLRNLTILDLSNNNIANINDDMLEGLEKLEILDLQHNNLARLWKHANPGGPIYFLKGLSHLHILNLESNGFDEIPVEVFKDLFELKIIDLGLNNLNTLPASVFNNQVSLKSLNLQKNLITSVEKKVFGPAFRNLTELDMRFNPFDCTCESIAWFVNWINETHTNIPELSSHYLCNTPPHYHGFPVRLFDTSSCKDSAPFELFFMINTSILLIFIFIVLLIHFEGWRISFYWNVSVHRVLGFKEIDRQTEQFEYAAYIIHAYKDKDWVWEHFSSMEKEDQSLKFCLEERDFEAGVFELEAIVNSIKRSRKIIFVITHHLLKDPLCKRFKVHHAVQQAIEQNLDSIILVFLEEIPDYKLNHALCLRRGMFKSHCILNWPVQKERIGAFRHKLQVALGSKNSVH

SEQ ID NO: 3 - DNA, 2109 bp, Homo sapiens
atgagacagactttgccttgtatctacttttgggggggccttttgccctttgggatgctgtgtgcatcctccaccaccaagtgcactgttagccatgaagttgctgactgcagccacctgaagttgactcaggtacccgatgatctacccacaaacataacagtgttgaaccttacccataatcaactcagaagattaccagccgccaacttcacaaggtatagccagctaactagcttggatgtaggatttaacaccatctcaaaactggagccagaattgtgccagaaacttcccatgttaaaagttttgaacctccagcacaatgagctatctcaactttctgataaaacctttgccttctgcacgaatttgactgaactccatctcatgtccaactcaatccagaaaattaaaaataatccctttgtcaagcagaagaatttaatcacattagatctgtctcataatggcttgtcatctacaaaattaggaactcaggttcagctggaaaatctccaagagcttctattatcaaacaataaaattcaagcgctaaaaagtgaagaactggatatctttgccaattcatctttaaaaaaattagagttgtcatcgaatcaaattaaagagttttctccagggtgttttcacgcaattggaagattatttggcctctttctgaacaatgtccagctgggtcccagccttacagagaagctatgtttggaattagcaaacacaagcattcggaatctgtctctgagtaacagccagctgtccaccaccagcaatacaactttcttgggactaaagtggacaaatctcactatgctcgatctttcctacaacaacttaaatgtggttggtaacgattcctttgcttggcttccacaactagaatatttcttcctagagtataataatatacagcatttgttttctcactctttgcacgggcttttcaatgtgaggtacctgaatttgaaacggtcttttactaaacaaagtatttcccttgcctcactccccaagattgatgatttttcttttcagtggctaaaatgtttggagcaccttaacatggaagataatgatattccaggcataaaaagcaatatgttcacaggattgataaacctgaaatacttaagtctatccaactcctttacaagtttgcgaactttgacaaatgaaacatttgtatcacttgctcattctcccttacacatactcaacctaaccaagaataaaatctcaaaaatagagagtgatgctttctcttggttgggccacctagaagtacttgacctgggccttaatgaaattgggcaagaactcacaggccaggaatggagaggtctagaaaatattttcgaaatctatctttcctacaacaagtacctgcagctgactaggaactcctttgccttggtcccaagccttcaacgactgatgctccgaagggtggcccttaaaaatgtggatagctctccttcaccattccagcctcttcgtaacttgaccattctggatctaagcaacaacaacatagccaacataaatgatgacatgttggagggtcttgagaaactagaaattctcgatttgcagcataacaacttagcacggctctggaaacacgcaaaccctggtggtcccatttatttcctaaagggtctgtctcacctccacatccttaacttggagtccaacggctttgacgagatcccagttgaggtcttcaaggatttatttgaactaaagatcatcgatttaggattgaataatttaaacacacttccagcatctgtctttaataatcaggtgtctctaaagtcattgaaccttcagaagaatctcataacatccgttgagaagaaggttttcgggccagctttcaggaacctgactgagttagatatgcgctttaatccctttgattgcacgtgtgaaagtattgcctggtttgttaattggattaacgagacccataccaacatccctgagctgtcaagccactacctttgcaacactccacctcactatcatgggttcccagtgagactttttgatacatcatcttgcaaagacagtgccccctttgaa

SEQ ID NO: 4 - protein, 703 aa, Homo sapiens
MRQTLPCIYFWGGLLPFGMLCASSTTKCTVSHEVADCSHLKLTQVPDDLPTNITVLNLTHNQLRRLPAANFTRYSQLTSLDVGFNTISKLEPELCQKLPMLKVLNLQHNELSQLSDKTFAFCTNLTELHLMSNSIQKIKNNPFVKQKNLITLDLSHNGLSSTKLGTQVQLENLQELLLSNNKIQALKSEELDIFANSSLKKLELSSNQIKEFSPGCFHAIGRLFGLFLNNVQLGPSLTEKLCLELANTSIRNLSLSNSQLSTTSNTTFLGLKWTNLTMLDLSYNNLNVVGNDSFAWLPQLEYFFLEYNNIQHLFSHSLHGLFNVRYLNLKRSFTKQSISLASLPKIDDFSFQWLKCLEHLNMEDNDIPGIKSNMFTGLINLKYLSLSNSFTSLRTLTNETFVSLAHSPLHILNLTKNKISKIESDAFSWLGHLEVLDLGLNEIGQELTGQEWRGLENIFEIYLSYNKYLQLTRNSFALVPSLQRLMLRRVALKNVDSSPSPFQPLRNLTILDLSNNNIANINDDMLEGLEKLEILDLQHNNLARLWKHANPGGPIYFLKGLSHLHILNLESNGFDEIPVEVFKDLFELKIIDLGLNNLNTLPASVFNNQVSLKSLNLQKNLITSVEKKVFGPAFRNLTELDMRFNPFDCTCESIAWFVNWINETHTNIPELSSHYLCNTPPHYHGFPVRLFDTSSCKDSAPFE

SEQ ID NO: 5 - DNA, 2031 bp, Homo sapiens
aagtgcactgttagccatgaagttgctgactgcagccacctgaagttgactcaggtacccgatgatctacccacaaacataacagtgttgaaccttacccataatcaactcagaagattaccagccgccaacttcacaaggtatagccagctaactagcttggatgtaggatttaacaccatctcaaaactggagccagaattgtgccagaaacttcccatgttaaaagttttgaacctccagcacaatgagctatctcaactttctgataaaacctttgccttctgcacgaatttgactgaactccatctcatgtccaactcaatccagaaaattaaaaataatccctttgtcaagcagaagaatttaatcacattagatctgtctcataatggcttgtcatctacaaaattaggaactcaggttcagctggaaaatctccaagagcttctattatcaaacaataaaattcaagcgctaaaaagtgaagaactggatatctttgccaattcatctttaaaaaaattagagttgtcatcgaatcaaattaaagagttttctccagggtgttttcacgcaattggaagattatttggcctctttctgaacaatgtccagctgggtcccagccttacagagaagctatgtttggaattagcaaacacaagcattcggaatctgtctctgagtaacagccagctgtccaccaccagcaatacaactttcttgggactaaagtggacaaatctcactatgctcgatctttcctacaacaacttaaatgtggttggtaacgattcctttgcttggcttccacaactagaatatttcttcctagagtataataatatacagcatttgttttctcactctttgcacgggcttttcaatgtgaggtacctgaatttgaaacggtcttttactaaacaaagtatttcccttgcctcactccccaagattgatgatttttcttttcagtggctaaaatgtttggagcaccttaacatggaagataatgatattccaggcataaaaagcaatatgttcacaggattgataaacctgaaatacttaagtctatccaactcctttacaagtttgcgaactttgacaaatgaaacatttgtatcacttgctcattctcccttacacatactcaacctaaccaagaataaaatctcaaaaatagagagtgatgctttctcttggttgggccacctagaagtacttgacctgggccttaatgaaattgggcaagaactcacaggccaggaatggagaggtctagaaaatattttcgaaatctatctttcctacaacaagtacctgcagctgactaggaactcctttgccttggtcccaagccttcaacgactgatgctccgaagggtggcccttaaaaatgtggatagctctccttcaccattccagcctcttcgtaacttgaccattctggatctaagcaacaacaacatagccaacataaatgatgacatgttggagggtcttgagaaactagaaattctcgatttgcagcataacaacttagcacggctctggaaacacgcaaaccctggtggtcccatttatttcctaaagggtctgtctcacctccacatccttaacttggagtccaacggctttgacgagatcccagttgaggtcttcaaggatttatttgaactaaagatcatcgatttaggattgaataatttaaacacacttccagcatctgtctttaataatcaggtgtctctaaagtcattgaaccttcagaagaatctcataacatccgttgagaagaaggttttcgggccagctttcaggaacctgactgagttagatatgcgctttaatccctttgattgcacgtgtgaaagtattgcctggtttgttaattggattaacgagacccataccaacatccctgagctgtcaagccactacctttgcaacactccacctcactatcatgggttcccagtgagactttttgatacatcatcttgcaaagacagtgccccctttgaa

SEQ ID NO: 6 - protein, 677 aa, Homo sapiens
KCTVSHEVADCSHLKLTQVPDDLPTNITVLNLTHNQLRRLPAANFTRYSQLTSLDVGFNTISKLEPELCQKLPMLKVLNLQHNELSQLSDKTFAFCTNLTELHLMSNSIQKIKNNPFVKQKNLITLDLSHNGLSSTKLGTQVQLENLQELLLSNNKIQALKSEELDIFANSSLKKLELSSNQIKEFSPGCFHAIGRLFGLFLNNVQLGPSLTEKLCLELANTSIRNLSLSNSQLSTTSNTTFLGLKWTNLTMLDLSYNNLNVVGNDSFAWLPQLEYFFLEYNNIQHLFSHSLHGLFNVRYLNLKRSFTKQSISLASLPKIDDFSFQWLKCLEHLNMEDNDIPGIKSNMFTGLINLKYLSLSNSFTSLRTLTNETFVSLAHSPLHILNLTKNKISKIESDAFSWLGHLEVLDLGLNEIGQELTGQEWRGLENIFEIYLSYNKYLQLTRNSFALVPSLQRLMLRRVALKNVDSSPSPFQPLRNLTILDLSNNNIANINDDMLEGLEKLEILDLQHNNLARLWKHANPGGPIYFLKGLSHLHILNLESNGFDEIPVEVFKDLFELKIIDLGLNNLNTLPASVFNNQVSLKSLNLQKNLITSVEKKVFGPAFRNLTELDMRFNPFDCTCESIAWFVNWINETHTNIPELSSHYLCNTPPHYHGFPVRLFDTSSCKDSAPFE

SEQ ID NO: 7 - DNA, 2031 bp, Artificial Sequence
  Artificial cDNA sequence derived from the Homo sapiens hTLR3 ECD which lacks a signal peptide and contains an alanine (A) substitution mutation at N221.
aaatgcaccgtgagccatgaagtggcggattgcagccatctgaaactgacccaggtgccggatgatctgccgaccaacattaccgtgctgaacctgacccataaccagctgcgccgcctgccggcggcgaactttacccgctatagccagctgaccagcctggatgtgggctttaacaccattagcaaactggaaccggaactgtgccagaaactgccgatgctgaaagtgctgaacctgcagcataacgaactgagccagctgagcgataaaacctttgcgttttgcaccaacctgaccgaactgcatctgatgagcaacagcattcagaaaattaaaaacaacccgtttgtgaaacagaaaaacctgattaccctggatctgagccataacggcctgagcagcaccaaactgggcacccaggtgcagctggaaaacctgcaggaactgctgctgagcaacaacaaaattcaggcgctgaaaagcgaagaactggatatttttgcgaacagcagcctgaaaaaactggaactgagcagcaaccagattaaagaatttagcccgggctgctttcatgcgattggccgcctgtttggcctgtttctgaacaacgtgcagctgggcccgagcctgaccgaaaaactgtgcctggaactggcggcgaccagcattcgcaacctgagcctgagcaacagccagctgagcaccaccagcaacaccacctttctgggcctgaaatggaccaacctgaccatgctggatctgagctataacaacctgaacgtggtgggcaacgatagctttgcgtggctgccgcagctggaatatttttttctggaatataacaacattcagcatctgtttagccatagcctgcatggcctgtttaacgtgcgctatctgaacctgaaacgcagctttaccaaacagagcattagcctggcgagcctgccgaaaattgatgattttagctttcagtggctgaaatgcctggaacatctgaacatggaagataacgatattccgggcattaaaagcaacatgtttaccggcctgattaacctgaaatatctgagcctgagcaacagctttaccagcctgcgcaccctgaccaacgaaacctttgtgagcctggcgcatagcccgctgcatattctgaacctgaccaaaaacaaaattagcaaaattgaaagcgatgcgtttagctggctgggccatctggaagtgctggatctgggcctgaacgaaattggccaggaactgaccggccaggaatggcgcggcctggaaaacatttttgaaatttatctgagctataacaaatatctgcagctgacccgcaacagctttgcgctggtgccgagcctgcagcgcctgatgctgcgccgcgtggcgctgaaaaacgtggatagcagcccgagcccgtttcagccgctgcgcaacctgaccattctggatctgagcaacaacaacattgcgaacattaacgatgatatgctggaaggcctggaaaaactggaaattctggatctgcagcataacaacctggcgcgcctgtggaaacatgcgaacccgggcggcccgatttattttctgaaaggcctgagccatctgcatattctgaacctggaaagcaacggctttgatgaaattccggtggaagtgtttaaagatctgtttgaactgaaaattattgatctgggcctgaacaacctgaacaccctgccggcgagcgtgtttaacaaccaggtgagcctgaaaagcctgaacctgcagaaaaacctgattaccagcgtggaaaaaaaagtgtttggcccggcgtttcgcaacctgaccgaactggatatgcgctttaacccgtttgattgcacctgcgaaagcattgcgtggtttgtgaactggattaacgaaacccataccaacattccggaactgagcagccattatctgtgcaacaccccgccgcattatcatggctttccggtgcgcctgtttgataccagcagctgcaaagatagcgcgccgtttgaa

SEQ ID NO: 8 - protein, 677 aa, Artificial Sequence
  Artificial amino acid sequence derived from the Homo sapiens hTLR3 ECD which lacks a signal peptide and contains an alanine (A) substitution mutation at N221.
KCTVSHEVADCSHLKLTQVPDDLPTNITVLNLTHNQLRRLPAANFTRYSQLTSLDVGFNTISKLEPELCQKLPMLKVLNLQHNELSQLSDKTFAFCTNLTELHLMSNSIQKIKNNPFVKQKNLITLDLSHNGLSSTKLGTQVQLENLQELLLSNNKIQALKSEELDIFANSSLKKLELSSNQIKEFSPGCFHAIGRLFGLFLNNVQLGPSLTEKLCLELAATSIRNLSLSNSQLSTTSNTTFLGLKWTNLTMLDLSYNNLNVVGNDSFAWLPQLEYFFLEYNNIQHLFSHSLHGLFNVRYLNLKRSFTKQSISLASLPKIDDFSFQWLKCLEHLNMEDNDIPGIKSNMFTGLINLKYLSLSNSFTSLRTLTNETFVSLAHSPLHILNLTKNKISKIESDAFSWLGHLEVLDLGLNEIGQELTGQEWRGLENIFEIYLSYNKYLQLTRNSFALVPSLQRLMLRRVALKNVDSSPSPFQPLRNLTILDLSNNNIANINDDMLEGLEKLEILDLQHNNLARLWKHANPGGPIYFLKGLSHLHILNLESNGFDEIPVEVFKDLFELKIIDLGLNNLNTLPASVFNNQVSLKSLNLQKNLITSVEKKVFGPAFRNLTELDMRFNPFDCTCESIAWFVNWINETHTNIPELSSHYLCNTPPHYHGFPVRLFDTSSCKDSAPFE

SEQ ID NO: 9 - DNA, 2031 bp, Artificial Sequence
  Artificial cDNA sequence derived from the Homo sapiens hTLR3 ECD which lacks a signal peptide and contains an alanine (A) substitution mutation at N387.
aaatgcaccgtgagccatgaagtggcggattgcagccatctgaaactgacccaggtgccggatgatctgccgaccaacattaccgtgctgaacctgacccataaccagctgcgccgcctgccggcggcgaactttacccgctatagccagctgaccagcctggatgtgggctttaacaccattagcaaactggaaccggaactgtgccagaaactgccgatgctgaaagtgctgaacctgcagcataacgaactgagccagctgagcgataaaacctttgcgttttgcaccaacctgaccgaactgcatctgatgagcaacagcattcagaaaattaaaaacaacccgtttgtgaaacagaaaaacctgattaccctggatctgagccataacggcctgagcagcaccaaactgggcacccaggtgcagctggaaaacctgcaggaactgctgctgagcaacaacaaaattcaggcgctgaaaagcgaagaactggatatttttgcgaacagcagcctgaaaaaactggaactgagcagcaaccagattaaagaatttagcccgggctgctttcatgcgattggccgcctgtttggcctgtttctgaacaacgtgcagctgggcccgagcctgaccgaaaaactgtgcctggaactggcgaacaccagcattcgcaacctgagcctgagcaacagccagctgagcaccaccagcaacaccacctttctgggcctgaaatggaccaacctgaccatgctggatctgagctataacaacctgaacgtggtgggcaacgatagctttgcgtggctgccgcagctggaatatttttttctggaatataacaacattcagcatctgtttagccatagcctgcatggcctgtttaacgtgcgctatctgaacctgaaacgcagctttaccaaacagagcattagcctggcgagcctgccgaaaattgatgattttagctttcagtggctgaaatgcctggaacatctgaacatggaagataacgatattccgggcattaaaagcaacatgtttaccggcctgattaacctgaaatatctgagcctgagcaacagctttaccagcctgcgcaccctgaccaacgaaacctttgtgagcctggcgcatagcccgctgcatattctggcgctgaccaaaaacaaaattagcaaaattgaaagcgatgcgtttagctggctgggccatctggaagtgctggatctgggcctgaacgaaattggccaggaactgaccggccaggaatggcgcggcctggaaaacatttttgaaatttatctgagctataacaaatatctgcagctgacccgcaacagctttgcgctggtgccgagcctgcagcgcctgatgctgcgccgcgtggcgctgaaaaacgtggatagcagcccgagcccgtttcagccgctgcgcaacctgaccattctggatctgagcaacaacaacattgcgaacattaacgatgatatgctggaaggcctggaaaaactggaaattctggatctgcagcataacaacctggcgcgcctgtggaaacatgcgaacccgggcggcccgatttattttctgaaaggcctgagccatctgcatattctgaacctggaaagcaacggctttgatgaaattccggtggaagtgtttaaagatctgtttgaactgaaaattattgatctgggcctgaacaacctgaacaccctgccggcgagcgtgtttaacaaccaggtgagcctgaaaagcctgaacctgcagaaaaacctgattaccagcgtggaaaaaaaagtgtttggcccggcgtttcgcaacctgaccgaactggatatgcgctttaacccgtttgattgcacctgcgaaagcattgcgtggtttgtgaactggattaacgaaacccataccaacattccggaactgagcagccattatctgtgcaacaccccgccgcattatcatggctttccggtgcgcctgtttgataccagcagctgcaaagatagcgcgccgtttgaa

SEQ ID NO: 10 - protein, 677 aa, Artificial Sequence
  Artificial amino acid sequence derived from the Homo sapiens hTLR3 ECD which lacks a signal peptide and contains an alanine (A) substitution mutation at N387.
KCTVSHEVADCSHLKLTQVPDDLPTNITVLNLTHNQLRRLPAANFTRYSQLTSLDVGFNTISKLEPELCQKLPMLKVLNLQHNELSQLSDKTFAFCTNLTELHLMSNSIQKIKNNPFVKQKNLITLDLSHNGLSSTKLGTQVQLENLQELLLSNNKIQALKSEELDIFANSSLKKLELSSNQIKEFSPGCFHAIGRLFGLFLNNVQLGPSLTEKLCLELANTSIRNLSLSNSQLSTTSNTTFLGLKWTNLTMLDLSYNNLNVVGNDSFAWLPQLEYFFLEYNNIQHLFSHSLHGLFNVRYLNLKRSFTKQSISLASLPKIDDFSFQWLKCLEHLNMEDNDIPGIKSNMFTGLINLKYLSLSNSFTSLRTLTNETFVSLAHSPLHILALTKNKISKIESDAFSWLGHLEVLDLGLNEIGQELTGQEWRGLENIFEIYLSYNKYLQLTRNSFALVPSLQRLMLRRVALKNVDSSPSPFQPLRNLTILDLSNNNIANINDDMLEGLEKLEILDLQHNNLARLWKHANPGGPIYFLKGLSHLHILNLESNGFDEIPVEVFKDLFELKIIDLGLNNLNTLPASVFNNQVSLKSLNLQKNLITSVEKKVFGPAFRNLTELDMRFNPFDCTCESIAWFVNWINETHTNIPELSSHYLCNTPPHYHGFPVRLFDTSSCKDSAPFE

SEQ ID NO: 11 - DNA, 2031 bp, Artificial Sequence
  Artificial cDNA sequence derived from the Homo sapiens hTLR3 ECD which lacks a signal peptide and contains alanine (A) substitution mutations at N221 and N387.
aaatgcaccgtgagccatgaagtggcggattgcagccatctgaaactgacccaggtgccggatgatctgccgaccaacattaccgtgctgaacctgacccataaccagctgcgccgcctgccggcggcgaactttacccgctatagccagctgaccagcctggatgtgggctttaacaccattagcaaactggaaccggaactgtgccagaaactgccgatgctgaaagtgctgaacctgcagcataacgaactgagccagctgagcgataaaacctttgcgttttgcaccaacctgaccgaactgcatctgatgagcaacagcattcagaaaattaaaaacaacccgtttgtgaaacagaaaaacctgattaccctggatctgagccataacggcctgagcagcaccaaactgggcacccaggtgcagctggaaaacctgcaggaactgctgctgagcaacaacaaaattcaggcgctgaaaagcgaagaactggatatttttgcgaacagcagcctgaaaaaactggaactgagcagcaaccagattaaagaatttagcccgggctgctttcatgcgattggccgcctgtttggcctgtttctgaacaacgtgcagctgggcccgagcctgaccgaaaaactgtgcctggaactggcggcgaccagcattcgcaacctgagcctgagcaacagccagctgagcaccaccagcaacaccacctttctgggcctgaaatggaccaacctgaccatgctggatctgagctataacaacctgaacgtggtgggcaacgatagctttgcgtggctgccgcagctggaatatttttttctggaatataacaacattcagcatctgtttagccatagcctgcatggcctgtttaacgtgcgctatctgaacctgaaacgcagctttaccaaacagagcattagcctggcgagcctgccgaaaattgatgattttagctttcagtggctgaaatgcctggaacatctgaacatggaagataacgatattccgggcattaaaagcaacatgtttaccggcctgattaacctgaaatatctgagcctgagcaacagctttaccagcctgcgcaccctgaccaacgaaacctttgtgagcctggcgcatagcccgctgcatattctggcgctgaccaaaaacaaaattagcaaaattgaaagcgatgcgtttagctggctgggccatctggaagtgctggatctgggcctgaacgaaattggccaggaactgaccggccaggaatggcgcggcctggaaaacatttttgaaatttatctgagctataacaaatatctgcagctgacccgcaacagctttgcgctggtgccgagcctgcagcgcctgatgctgcgccgcgtggcgctgaaaaacgtggatagcagcccgagcccgtttcagccgctgcgcaacctgaccattctggatctgagcaacaacaacattgcgaacattaacgatgatatgctggaaggcctggaaaaactggaaattctggatctgcagcataacaacctggcgcgcctgtggaaacatgcgaacccgggcggcccgatttattttctgaaaggcctgagccatctgcatattctgaacctggaaagcaacggctttgatgaaattccggtggaagtgtttaaagatctgtttgaactgaaaattattgatctgggcctgaacaacctgaacaccctgccggcgagcgtgtttaacaaccaggtgagcctgaaaagcctgaacctgcagaaaaacctgattaccagcgtggaaaaaaaagtgtttggcccggcgtttcgcaacctgaccgaactggatatgcgctttaacccgtttgattgcacctgcgaaagcattgcgtggtttgtgaactggattaacgaaacccataccaacattccggaactgagcagccattatctgtgcaacaccccgccgcattatcatggctttccggtgcgcctgtttgataccagcagctgcaaagatagcgcgccgtttgaa

SEQ ID NO: 12 - protein, 677 aa, Artificial Sequence
  Artificial amino acid sequence derived from the Homo sapiens hTLR3 ECD which lacks a signal peptide and contains alanine (A) substitution mutations at N221 and N387.
KCTVSHEVADCSHLKLTQVPDDLPTNITVLNLTHNQLRRLPAANFTRYSQLTSLDVGFNTISKLEPELCQKLPMLKVLNLQHNELSQLSDKTFAFCTNLTELHLMSNSIQKIKNNPFVKQKNLITLDLSHNGLSSTKLGTQVQLENLQELLLSNNKIQALKSEELDIFANSSLKKLELSSNQIKEFSPGCFHAIGRLFGLFLNNVQLGPSLTEKLCLELAATSIRNLSLSNSQLSTTSNTTFLGLKWTNLTMLDLSYNNLNVVGNDSFAWLPQLEYFFLEYNNIQHLFSHSLHGLFNVRYLNLKRSFTKQSISLASLPKIDDFSFQWLKCLEHLNMEDNDIPGIKSNMFTGLINLKYLSLSNSFTSLRTLTNETFVSLAHSPLHILALTKNKISKIESDAFSWLGHLEVLDLGLNEIGQELTGQEWRGLENIFEIYLSYNKYLQLTRNSFALVPSLQRLMLRRVALKNVDSSPSPFQPLRNLTILDLSNNNIANINDDMLEGLEKLEILDLQHNNLARLWKHANPGGPIYFLKGLSHLHILNLESNGFDEIPVEVFKDLFELKIIDLGLNNLNTLPASVFNNQVSLKSLNLQKNLITSVEKKVFGPAFRNLTELDMRFNPFDCTCESIAWFVNWINETHTNIPELSSHYLCNTPPHYHGFPVRLFDTSSCKDSAPFE

SEQ ID NO: 13 - DNA, 2031 bp, Artificial Sequence
  Artificial cDNA sequence derived from the Mus musculus TLR3 ECD which lacks a signal peptide and contains alanine (A) substitution mutations at N221, N226, N387, and N636.
cagtgcaccgtgcgctataacgtggcggattgcagccatctgaaactgacccatattccggatgatctgccgagcaacattaccgtgctgaacctgacccataaccagctgcgccgcctgccgccgaccaactttacccgctatagccagctggcgattctggatgcgggctttaacagcattagcaaactggaaccggaactgtgccagattctgccgctgctgaaagtgctgaacctgcagcataacgaactgagccagattagcgatcagacctttgtgttttgcaccaacctgaccgaactggatctgatgagcaacagcattcataaaattaaaagcaacccgtttaaaaaccagaaaaacctgattaaactggatctgagccataacggcctgagcagcaccaaactgggcaccggcgtgcagctggaaaacctgcaggaactgctgctggcgaaaaacaaaattctggcgctgcgcagcgaagaactggaatttctgggcaacagcagcctgcgcaaactggatctgagcagcaacccgctgaaagaatttagcccgggctgctttcagaccattggcaaactgtttgcgctgctgctgaacaacgcgcagctgaacccgcatctgaccgaaaaactgtgctgggaactgagcgcgaccagcattcaggcgctgagcctggcgaacaaccagctgctggcgaccagcgaaagcacctttagcggcctgaaatggaccaacctgacccagctggatctgagctataacaacctgcatgatgtgggcaacggcagctttagctatctgccgagcctgcgctatctgagcctggaatataacaacattcagcgcctgagcccgcgcagcttttatggcctgagcaacctgcgctatctgagcctgaaacgcgcgtttaccaaacagagcgtgagcctggcgagccatccgaacattgatgattttagctttcagtggctgaaatatctggaatatctgaacatggatgataacaacattccgagcaccaaaagcaacacctttaccggcctggtgagcctgaaatatctgagcctgagcaaaacctttaccagcctgcagaccctgaccaacgaaacctttgtgagcctggcgcatagcccgctgctgaccctggcgctgaccaaaaaccatattagcaaaattgcgaacggcacctttagctggctgggccagctgcgcattctggatctgggcctgaacgaaattgaacagaaactgagcggccaggaatggcgcggcctgcgcaacatttttgaaatttatctgagctataacaaatatctgcagctgagcaccagcagctttgcgctggtgccgagcctgcagcgcctgatgctgcgccgcgtggcgctgaaaaacgtggatattagcccgagcccgtttcgcccgctgcgcaacctgaccattctggatctgagcaacaacaacattgcgaacattaacgaagatctgctggaaggcctggaaaacctggaaattctggattttcagcataacaacctggcgcgcctgtggaaacgcgcgaacccgggcggcccggtgaactttctgaaaggcctgagccatctgcatattctgaacctggaaagcaacggcctggatgaaattccggtgggcgtgtttaaaaacctgtttgaactgaaaagcattaacctgggcctgaacaacctgaacaaactggaaccgtttatttttgatgatcagaccagcctgcgcagcctgaacctgcagaaaaacctgattaccagcgtggaaaaagatgtgtttggcccgccgtttcagaacctgaacagcctggatatgcgctttaacccgtttgattgcacctgcgaaagcattagctggtttgtgaactggattgcgcagacccataccaacattagcgaactgagcacccattatctgtgcaacaccccgcatcattattatggctttccgctgaaactgtttgataccagcagctgcaaagatagcgcgccgtttgaa

SEQ ID NO: 14 - protein, 677 aa, Artificial Sequence
  Artificial amino acid sequence derived from the Mus musculus TLR3 ECD which lacks a signal peptide and contains alanine (A) substitution mutations at N221, N226, N387, and N636.
QCTVRYNVADCSHLKLTHIPDDLPSNITVLNLTHNQLRRLPPTNFTRYSQLAILDAGFNSISKLEPELCQILPLLKVLNLQHNELSQISDQTFVFCTNLTELDLMSNSIHKIKSNPFKNQKNLIKLDLSHNGLSSTKLGTGVQLENLQELLLAKNKILALRSEELEFLGNSSLRKLDLSSNPLKEFSPGCFQTIGKLFALLLNNAQLNPHLTEKLCWELSATSIQALSLANNQLLATSESTFSGLKWTNLTQLDLSYNNLHDVGNGSFSYLPSLRYLSLEYNNIQRLSPRSFYGLSNLRYLSLKRAFTKQSVSLASHPNIDDFSFQWLKYLEYLNMDDNNIPSTKSNTFTGLVSLKYLSLSKTFTSLQTLTNETFVSLAHSPLLTLALTKNHISKIANGTFSWLGQLRILDLGLNEIEQKLSGQEWRGLRNIFEIYLSYNKYLQLSTSSFALVPSLQRLMLRRVALKNVDISPSPFRPLRNLTILDLSNNNIANINEDLLEGLENLEILDFQHNNLARLWKRANPGGPVNFLKGLSHLHILNLESNGLDEIPVGVFKNLFELKSINLGLNNLNKLEPFIFDDQTSLRSLNLQKNLITSVEKDVFGPPFQNLNSLDMRFNPFDCTCESISWFVNWIAQTHTNISELSTHYLCNTPHHYYGFPLKLFDTSSCKDSAPFE

SEQ ID NO: 15 - DNA, 2031 bp, Artificial Sequence
  Artificial cDNA sequence derived from the Homo sapiens hTLR3 ECD which lacks a signal peptide and contains an alanine (A) substitution mutation at N226.
aaatgcaccgtgagccatgaagtggcggattgcagccatctgaaactgacccaggtgccggatgatctgccgaccaacattaccgtgctgaacctgacccataaccagctgcgccgcctgccggcggcgaactttacccgctatagccagctgaccagcctggatgtgggctttaacaccattagcaaactggaaccggaactgtgccagaaactgccgatgctgaaagtgctgaacctgcagcataacgaactgagccagctgagcgataaaacctttgcgttttgcaccaacctgaccgaactgcatctgatgagcaacagcattcagaaaattaaaaacaacccgtttgtgaaacagaaaaacctgattaccctggatctgagccataacggcctgagcagcaccaaactgggcacccaggtgcagctggaaaacctgcaggaactgctgctgagcaacaacaaaattcaggcgctgaaaagcgaagaactggatatttttgcgaacagcagcctgaaaaaactggaactgagcagcaaccagattaaagaatttagcccgggctgctttcatgcgattggccgcctgtttggcctgtttctgaacaacgtgcagctgggcccgagcctgaccgaaaaactgtgcctggaactggcgaacaccagcattcgcgcgctgagcctgagcaacagccagctgagcaccaccagcaacaccacctttctgggcctgaaatggaccaacctgaccatgctggatctgagctataacaacctgaacgtggtgggcaacgatagctttgcgtggctgccgcagctggaatatttttttctggaatataacaacattcagcatctgtttagccatagcctgcatggcctgtttaacgtgcgctatctgaacctgaaacgcagctttaccaaacagagcattagcctggcgagcctgccgaaaattgatgattttagctttcagtggctgaaatgcctggaacatctgaacatggaagataacgatattccgggcattaaaagcaacatgtttaccggcctgattaacctgaaatatctgagcctgagcaacagctttaccagcctgcgcaccctgaccaacgaaacctttgtgagcctggcgcatagcccgctgcatattctgaacctgaccaaaaacaaaattagcaaaattgaaagcgatgcgtttagctggctgggccatctggaagtgctggatctgggcctgaacgaaattggccaggaactgaccggccaggaatggcgcggcctggaaaacatttttgaaatttatctgagctataacaaatatctgcagctgacccgcaacagctttgcgctggtgccgagcctgcagcgcctgatgctgcgccgcgtggcgctgaaaaacgtggatagcagcccgagcccgtttcagccgctgcgcaacctgaccattctggatctgagcaacaacaacattgcgaacattaacgatgatatgctggaaggcctggaaaaactggaaattctggatctgcagcataacaacctggcgcgcctgtggaaacatgcgaacccgggcggcccgatttattttctgaaaggcctgagccatctgcatattctgaacctggaaagcaacggctttgatgaaattccggtggaagtgtttaaagatctgtttgaactgaaaattattgatctgggcctgaacaacctgaacaccctgccggcgagcgtgtttaacaaccaggtgagcctgaaaagcctgaacctgcagaaaaacctgattaccagcgtggaaaaaaaagtgtttggcccggcgtttcgcaacctgaccgaactggatatgcgctttaacccgtttgattgcacctgcgaaagcattgcgtggtttgtgaactggattaacgaaacccataccaacattccggaactgagcagccattatctgtgcaacaccccgccgcattatcatggctttccggtgcgcctgtttgataccagcagctgcaaagatagcgcgccgtttgaa

SEQ ID NO: 16 - protein, 677 aa, Artificial Sequence
  Artificial amino acid sequence derived from the Homo sapiens hTLR3 ECD which lacks a signal peptide and contains an alanine (A) substitution mutation at N226.
KCTVSHEVADCSHLKLTQVPDDLPTNITVLNLTHNQLRRLPAANFTRYSQLTSLDVGFNTISKLEPELCQKLPMLKVLNLQHNELSQLSDKTFAFCTNLTELHLMSNSIQKIKNNPFVKQKNLITLDLSHNGLSSTKLGTQVQLENLQELLLSNNKIQALKSEELDIFANSSLKKLELSSNQIKEFSPGCFHAIGRLFGLFLNNVQLGPSLTEKLCLELANTSIRALSLSNSQLSTTSNTTFLGLKWTNLTMLDLSYNNLNVVGNDSFAWLPQLEYFFLEYNNIQHLFSHSLHGLFNVRYLNLKRSFTKQSISLASLPKIDDFSFQWLKCLEHLNMEDNDIPGIKSNMFTGLINLKYLSLSNSFTSLRTLTNETFVSLAHSPLHILNLTKNKISKIESDAFSWLGHLEVLDLGLNEIGQELTGQEWRGLENIFEIYLSYNKYLQLTRNSFALVPSLQRLMLRRVALKNVDSSPSPFQPLRNLTILDLSNNNIANINDDMLEGLEKLEILDLQHNNLARLWKHANPGGPIYFLKGLSHLHILNLESNGFDEIPVEVFKDLFELKIIDLGLNNLNTLPASVFNNQVSLKSLNLQKNLITSVEKKVFGPAFRNLTELDMRFNPFDCTCESIAWFVNWINETHTNIPELSSHYLCNTPPHYHGFPVRLFDTSSCKDSAPFE

SEQ ID NO: 17 - DNA, 2031 bp, Artificial Sequence
  Artificial cDNA sequence derived from the Homo sapiens hTLR3 ECD which lacks a signal peptide and contains an alanine (A) substitution mutation at N636.
aaatgcaccgtgagccatgaagtggcggattgcagccatctgaaactgacccaggtgccggatgatctgccgaccaacattaccgtgctgaacctgacccataaccagctgcgccgcctgccggcggcgaactttacccgctatagccagctgaccagcctggatgtgggctttaacaccattagcaaactggaaccggaactgtgccagaaactgccgatgctgaaagtgctgaacctgcagcataacgaactgagccagctgagcgataaaacctttgcgttttgcaccaacctgaccgaactgcatctgatgagcaacagcattcagaaaattaaaaacaacccgtttgtgaaacagaaaaacctgattaccctggatctgagccataacggcctgagcagcaccaaactgggcacccaggtgcagctggaaaacctgcaggaactgctgctgagcaacaacaaaattcaggcgctgaaaagcgaagaactggatatttttgcgaacagcagcctgaaaaaactggaactgagcagcaaccagattaaagaatttagcccgggctgctttcatgcgattggccgcctgtttggcctgtttctgaacaacgtgcagctgggcccgagcctgaccgaaaaactgtgcctggaactggcgaacaccagcattcgcaacctgagcctgagcaacagccagctgagcaccaccagcaacaccacctttctgggcctgaaatggaccaacctgaccatgctggatctgagctataacaacctgaacgtggtgggcaacgatagctttgcgtggctgccgcagctggaatatttttttctggaatataacaacattcagcatctgtttagccatagcctgcatggcctgtttaacgtgcgctatctgaacctgaaacgcagctttaccaaacagagcattagcctggcgagcctgccgaaaattgatgattttagctttcagtggctgaaatgcctggaacatctgaacatggaagataacgatattccgggcattaaaagcaacatgtttaccggcctgattaacctgaaatatctgagcctgagcaacagctttaccagcctgcgcaccctgaccaacgaaacctttgtgagcctggcgcatagcccgctgcatattctgaacctgaccaaaaacaaaattagcaaaattgaaagcgatgcgtttagctggctgggccatctggaagtgctggatctgggcctgaacgaaattggccaggaactgaccggccaggaatggcgcggcctggaaaacatttttgaaatttatctgagctataacaaatatctgcagctgacccgcaacagctttgcgctggtgccgagcctgcagcgcctgatgctgcgccgcgtggcgctgaaaaacgtggatagcagcccgagcccgtttcagccgctgcgcaacctgaccattctggatctgagcaacaacaacattgcgaacattaacgatgatatgctggaaggcctggaaaaactggaaattctggatctgcagcataacaacctggcgcgcctgtggaaacatgcgaacccgggcggcccgatttattttctgaaaggcctgagccatctgcatattctgaacctggaaagcaacggctttgatgaaattccggtggaagtgtttaaagatctgtttgaactgaaaattattgatctgggcctgaacaacctgaacaccctgccggcgagcgtgtttaacaaccaggtgagcctgaaaagcctgaacctgcagaaaaacctgattaccagcgtggaaaaaaaagtgtttggcccggcgtttcgcaacctgaccgaactggatatgcgctttaacccgtttgattgcacctgcgaaagcattgcgtggtttgtgaactggattgcggaaacccataccaacattccggaactgagcagccattatctgtgcaacaccccgccgcattatcatggctttccggtgcgcctgtttgataccagcagctgcaaagatagcgcgccgtttgaa

SEQ ID NO: 18 - protein, 677 aa, Artificial Sequence
  Artificial amino acid sequence derived from the Homo sapiens hTLR3 ECD which lacks a signal peptide and contains an alanine (A) substitution mutation at N636.
KCTVSHEVADCSHLKLTQVPDDLPTNITVLNLTHNQLRRLPAANFTRYSQLTSLDVGFNTISKLEPELCQKLPMLKVLNLQHNELSQLSDKTFAFCTNLTELHLMSNSIQKIKNNPFVKQKNLITLDLSHNGLSSTKLGTQVQLENLQELLLSNNKIQALKSEELDIFANSSLKKLELSSNQIKEFSPGCFHAIGRLFGLFLNNVQLGPSLTEKLCLELANTSIRNLSLSNSQLSTTSNTTFLGLKWTNLTMLDLSYNNLNVVGNDSFAWLPQLEYFFLEYNNIQHLFSHSLHGLFNVRYLNLKRSFTKQSISLASLPKIDDFSFQWLKCLEHLNMEDNDIPGIKSNMFTGLINLKYLSLSNSFTSLRTLTNETFVSLAHSPLHILNLTKNKISKIESDAFSWLGHLEVLDLGLNEIGQELTGQEWRGLENIFEIYLSYNKYLQLTRNSFALVPSLQRLMLRRVALKNVDSSPSPFQPLRNLTILDLSNNNIANINDDMLEGLEKLEILDLQHNNLARLWKHANPGGPIYFLKGLSHLHILNLESNGFDEIPVEVFKDLFELKIIDLGLNNLNTLPASVFNNQVSLKSLNLQKNLITSVEKKVFGPAFRNLTELDMRFNPFDCTCESIAWFVNWIAETHTNIPELSSHYLCNTPPHYHGFPVRLFDTSSCKDSAPFE

SEQ ID NO: 19 - DNA, 2031 bp, Artificial Sequence
  Artificial cDNA sequence derived from the Homo sapiens hTLR3 ECD which lacks a signal peptide and contains an alanine (A) substitution mutations at N221, N226, N387, and N636.
aaatgcaccgtgagccatgaagtggcggattgcagccatctgaaactgacccaggtgccggatgatctgccgaccaacattaccgtgctgaacctgacccataaccagctgcgccgcctgccggcggcgaactttacccgctatagccagctgaccagcctggatgtgggctttaacaccattagcaaactggaaccggaactgtgccagaaactgccgatgctgaaagtgctgaacctgcagcataacgaactgagccagctgagcgataaaacctttgcgttttgcaccaacctgaccgaactgcatctgatgagcaacagcattcagaaaattaaaaacaacccgtttgtgaaacagaaaaacctgattaccctggatctgagccataacggcctgagcagcaccaaactgggcacccaggtgcagctggaaaacctgcaggaactgctgctgagcaacaacaaaattcaggcgctgaaaagcgaagaactggatatttttgcgaacagcagcctgaaaaaactggaactgagcagcaaccagattaaagaatttagcccgggctgctttcatgcgattggccgcctgtttggcctgtttctgaacaacgtgcagctgggcccgagcctgaccgaaaaactgtgcctggaactggcggcgaccagcattcgcgcgctgagcctgagcaacagccagctgagcaccaccagcaacaccacctttctgggcctgaaatggaccaacctgaccatgctggatctgagctataacaacctgaacgtggtgggcaacgatagctttgcgtggctgccgcagctggaatatttttttctggaatataacaacattcagcatctgtttagccatagcctgcatggcctgtttaacgtgcgctatctgaacctgaaacgcagctttaccaaacagagcattagcctggcgagcctgccgaaaattgatgattttagctttcagtggctgaaatgcctggaacatctgaacatggaagataacgatattccgggcattaaaagcaacatgtttaccggcctgattaacctgaaatatctgagcctgagcaacagctttaccagcctgcgcaccctgaccaacgaaacctttgtgagcctggcgcatagcccgctgcatattctggcgctgaccaaaaacaaaattagcaaaattgaaagcgatgcgtttagctggctgggccatctggaagtgctggatctgggcctgaacgaaattggccaggaactgaccggccaggaatggcgcggcctggaaaacatttttgaaatttatctgagctataacaaatatctgcagctgacccgcaacagctttgcgctggtgccgagcctgcagcgcctgatgctgcgccgcgtggcgctgaaaaacgtggatagcagcccgagcccgtttcagccgctgcgcaacctgaccattctggatctgagcaacaacaacattgcgaacattaacgatgatatgctggaaggcctggaaaaactggaaattctggatctgcagcataacaacctggcgcgcctgtggaaacatgcgaacccgggcggcccgatttattttctgaaaggcctgagccatctgcatattctgaacctggaaagcaacggctttgatgaaattccggtggaagtgtttaaagatctgtttgaactgaaaattattgatctgggcctgaacaacctgaacaccctgccggcgagcgtgtttaacaaccaggtgagcctgaaaagcctgaacctgcagaaaaacctgattaccagcgtggaaaaaaaagtgtttggcccggcgtttcgcaacctgaccgaactggatatgcgctttaacccgtttgattgcacctgcgaaagcattgcgtggtttgtgaactggattgcggaaacccataccaacattccggaactgagcagccattatctgtgcaacaccccgccgcattatcatggctttccggtgcgcctgtttgataccagcagctgcaaagatagcgcgccgtttgaa

SEQ ID NO: 20 - protein, 677 aa, Artificial Sequence
  Artificial amino acid sequence derived from the Homo sapiens hTLR3 ECD which lacks a signal peptide and contains an alanine (A) substitution mutations at N221, N226, N387, and N636.
KCTVSHEVADCSHLKLTQVPDDLPTNITVLNLTHNQLRRLPAANFTRYSQLTSLDVGFNTISKLEPELCQKLPMLKVLNLQHNELSQLSDKTFAFCTNLTELHLMSNSIQKIKNNPFVKQKNLITLDLSHNGLSSTKLGTQVQLENLQELLLSNNKIQALKSEELDIFANSSLKKLELSSNQIKEFSPGCFHAIGRLFGLFLNNVQLGPSLTEKLCLELAATSIRALSLSNSQLSTTSNTTFLGLKWTNLTMLDLSYNNLNVVGNDSFAWLPQLEYFFLEYNNIQHLFSHSLHGLFNVRYLNLKRSFTKQSISLASLPKIDDFSFQWLKCLEHLNMEDNDIPGIKSNMFTGLINLKYLSLSNSFTSLRTLTNETFVSLAHSPLHILALTKNKISKIESDAFSWLGHLEVLDLGLNEIGQELTGQEWRGLENIFEIYLSYNKYLQLTRNSFALVPSLQRLMLRRVALKNVDSSPSPFQPLRNLTILDLSNNNIANINDDMLEGLEKLEILDLQHNNLARLWKHANPGGPIYFLKGLSHLHILNLESNGFDEIPVEVFKDLFELKIIDLGLNNLNTLPASVFNNQVSLKSLNLQKNLITSVEKKVFGPAFRNLTELDMRFNPFDCTCESIAWFVNWIAETHTNIPELSSHYLCNTPPHYHGFPVRLFDTSSCKDSAPFE

(19) United States
(12) Patent Application Publication
(10) Pub. No.: US 2007/0203064 A1
(43) Pub. Date: Aug. 30, 2007

(54) TLR3 Glycosylation Site Muteins and Methods of Use

(76) Inventors: Karen E. Duffy, Trappe, PA (US); Mark Cunningham, Kennett Square, PA (US); M. Lamine Mbow, Carlsbad, CA (US); Robert T. Sarisky, Lansdale, PA (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 11/554,253

(22) Filed: Oct. 30, 2006

Related U.S. Application Data
(60) Provisional application No. 60/731,105, filed on Oct. 28, 2005.

(51) Int. Cl. A61K 38/17; A61K 48/00; C07K 14/705

(52) U.S. Cl. 514/12; 530/395; 514/44; 536/23.5; 514/35

(57) ABSTRACT

TLR3 glycosylation site muteins, nucleic acids encoding the muteins, and methods of modulating TLR3 activity in a cell are disclosed.

### CROSS-REFERENCE TO RELATED APPLICATIONS

This application claims priority to U.S. Provisional Application No. 60/731,105, filed 28 Oct. 2005, the entire contents of which is incorporated herein.

### FIELD OF THE INVENTION

The present invention relates to TLR3 glycosylation site muteins, nucleic acids encoding the muteins, and methods of modulating TLR3 activity in a cell.

### BACKGROUND OF THE INVENTION

Pathologies associated with inflammatory conditions represent a significant challenge in health care and can be painful, debilitating and lethal. For example, sepsis and sepsis-associated conditions affect more than 750,000 people annually in the U.S. with mortality rates of 28-50%, resulting in 215,000 annual deaths (Natanson et al., Crit. Care Med. 26:1927-1931 (1998); Angus et al., Crit. Care Med. 29:1303-1310 (2001)). Other inflammatory conditions such as the inflammatory bowel diseases (IBD) Crohn's disease and ulcerative colitis affect more than 1 million people per year in the U.S. (Hanauer et al., Rev. Gastroenterol. Disord. 3:81-92 (2003)).

Inflammatory pulmonary conditions affecting lung function such as chronic obstructive pulmonary disease (COPD), asthma and lung infections also affect significant numbers of people in the U.S. COPD, for example, affects an estimated 10 million adult Americans and the prevalence is rising (Mapel et al., Manag. Care Interface 17:61-66 (2004)). Pathologies associated with these inflammatory conditions and exacerbations of these conditions have significant health and economic impacts.

Exacerbation in pulmonary diseases such as asthma and COPD is characterized by the worsening of symptoms and a decline in lung function. Viral infections are associated with exacerbations of many pulmonary diseases (Johnston, Am. J. Respir. Crit. Care Med. 152: S46-52 (1995); Bandi et al., FEMS Immunol. Med. Microbiol. 37: 69-75 (2003)) and are believed to be a major cause of exacerbations. Secretion of pro-inflammatory cytokines in the lungs following viral infection represents a crucial step in promoting the inflammatory response in various lung diseases (Gern et al., Am. J. Respir. Cell. Mol. Biol. 28:731-737 (2003); Panina-Bordignon et al., Curr. Opin. Pulm. Med. 9:104-110 (2003)).

Recognition of microbial antigens by the host immune system is mediated through innate immune receptors, whose activation represents an important step in the initiation of an inflammatory response. Toll-Like Receptors (TLR) represent a family of innate immune receptors that play a crucial role in mediating an immune response to foreign antigens. TLR3, for example, is a mammalian pattern recognition receptor that recognizes double-stranded (ds) RNA as well as the synthetic ds RNA analog poly-riboinosinic-ribocytidylic acid (poly I:C), (Alexopoulou et al., Nature 413: 732-238 (2001)). Moreover, TLR3 has been shown to recognize endogenous ligands such as mRNA released from necrotic cells (Kariko et al., J. Biol. Chem. 26: 12542-12550 (2004)) indicating that necrotic cell death at inflammation sites may contribute to activation of TLR3. A full-length human TLR3 amino acid sequence and encoding polynucleotide sequence is shown in SEQ ID NO: 1 and SEQ ID NO: 2, respectively.

Activation of TLR3 by the ds viral RNA analog poly(I:C) or by endogenous mRNA ligands induces secretion of pro-inflammatory cytokines and chemokines, a finding that indicates TLR3 activation modulates disease outcome during infection-associated inflammation. Thus, TLR3 ligation in vivo is thought to occur in the context of viral infection (Tabeta et al., Proc. Natl. Acad. Sci. USA 101:3516-3521 (2004)) or necrosis associated with inflammation (Kariko et al., J. Biol. Chem. 26: 12542-12550 (2004)). Overall, these data demonstrate that ligation of TLR3 initiates cascades of phosphorylation and transcriptional activation events that result in the production of numerous inflammatory cytokines that are thought to contribute to innate immunity (reviewed by Takeda and Akira, J. Derm. Sci. 34:73-82 (2004)). Further, these data suggest that sustained TLR3 activation can be a critical component in the modulation of infection-associated inflammatory diseases. Published data lend support to this hypothesis as shown by findings that associate over-production of pro-inflammatory cytokines with systemic inflammatory response syndrome, infection-associated acute cytokine storms (reviewed by Van Amersfoort et al., Clin. Microbiol. Rev. 16: 379-414 (2003)) and immune-mediated chronic conditions such as rheumatoid arthritis (reviewed by Miossec et al., Curr. Opin. Rheumatol. 16:218-222 (2004)) and inflammatory bowel diseases (reviewed by Ogata and Hibi, Curr. Pharm. Des. 9: 1107-1113 (2003)).

Importantly, it is becoming clear that TLR3 activity also plays a significant role in conditions such as inflammatory bowel disease symptoms, sepsis, cytokine, chemokine, and growth factor mediated lung pathologies and pulmonary inflammatory conditions resulting from increased inflammatory cell infiltration into lung tissues. However, it has been unclear what, if any, effect TLR3 glycosylation has on TLR3 activity and TLR3 activity mediated conditions.

Thus, a need exists to understand the effect of TLR3 glycosylation on TLR3 activity and exploit this information to develop compositions and methods that effectively modulate TLR3 activity.

### BRIEF DESCRIPTION OF THE DRAWINGS

FIG. 1 panel (A) shows MALDI-TOF mass spectra of hTLR3 extracellular domain (ECD) and panel (B) shows MALDI-TOF mass spectra of hTLR3 ECD treated with deglycosidases.

FIG. 2 shows the effect of N-glycosylation with tunicamycin onpoly(I:C) induced activation of hTLR3 signaling in HEK293 cells.

FIG. 3 shows the effect of ECD N-glycosylation by mutagenesis of N247, N252, or N413 in hTLR3 (SEQ ID NO: 2) on activation of hTLR3 signaling in cells. Panel (A) is an alignment of possible N-glycosylation sites from selected hTLR3 homologs; panels (B) and (C) are hTLR3 activation assays performed with the indicated hTLR3 mutants at 10 μg/ml (B) or 2.5 μg/ml (C) of the hTLR3 ligand poly(I:C).

FIG. 4 shows the effect of hTLR3 ECD N-glycosylation by mutagenesis of N247, N252, or N662 of hTLR (SEQ ID NO: 2) on poly(I:C) induced activation of hTLR3 signaling in cells.

### SUMMARY OF THE INVENTION

One aspect of the invention is a peptide chain comprising an amino acid sequence with at least 75% identity to the amino acid sequence of SEQ ID NO: 6 and at least one mutation within 3 amino acid residues of a position aligning to N221, N226, N387, or N636 of the amino acid sequence SEQ ID NO: 6.

Another aspect of the invention is a peptide chain comprising at least one mutation within 3 amino acid residues of position N221, N226, N387, or N636 of the amino acid sequence SEQ ID NO: 6.

Another aspect of the invention is a peptide chain comprising the amino acid sequence SEQ ID NO: 8, SEQ ID NO: 10, SEQ ID NO: 12, SEQ ID NO: 14, SEQ ID NO: 16, SEQ ID NO: 18, or SEQ ID NO: 20.

Another aspect of the invention is a method of modulating TLR3 activity in a cell comprising decreasing TLR3 glycosylation in the cell.

### DETAILED DESCRIPTION OF THE INVENTION

All publications, including but not limited to patents and patent applications, cited in this specification are herein incorporated by reference as though fully set forth.

The term “homolog” means protein sequences having between 40% and 100% sequence identity to a reference sequence. Homologs of hTLR3 include peptide chains from other species that have between 40% and 100% sequence identity to a known hTLR3 sequence. The terms “TLR3 homolog” and “TLR3” are used interchangeably throughout the specification and claims.

The term “peptide chain” as used herein means a molecule comprising at least two naturally or non-naturally occurring amino acid residues linked by peptide bonds.

The term “TLR3 activity” as used herein refers to any activities occurring as a result of ligand binding to a cell surface TLR3 homolog or that are mediated, in whole or in part, by at least one TLR3 homolog peptide chain.

The compositions and methods of the invention are useful in modulating the activity of TLR3 homologs in cells both in vitro and in vivo. In particular, the compositions and methods of the invention can be used to attenuate in vivo TLR3 dependent signaling and biological processes associated with TLR3 activity in conditions such as inflammatory bowel disease symptoms, sepsis, cytokine, chemokine, and growth factor mediated lung pathologies and pulmonary inflammatory conditions resulting from increased inflammatory cell infiltration into lung tissues.

One aspect of the invention is a peptide chain comprising an amino acid sequence with at least 75% identity to the amino acid sequence of SEQ ID NO: 6 and at least one mutation within 3 amino acid residues of a position aligning to N221, N226, N387, or N636 of the amino acid sequence SEQ ID NO: 6. Percent identity between two peptide chains can be determined by alignment using the default settings of the BLASTP 2.2.12 [Aug. 7, 2005] algorithm with low complexity filtering turned off and using SEQ ID NO: 6 as the BLASTP query sequence. The amino acid sequences of SEQ ID NO: 8, SEQ ID NO: 10, SEQ ID NO: 12, SEQ ID NO: 14, SEQ ID NO: 16, SEQ ID NO: 18, and SEQ ID NO: 20 are exemplary of such peptide chains. Such peptide chains may comprise additional sequences in addition to the mutagenized mature form ECD TLR3 homolog. Such additional sequences may be, for example, signal peptides such as a native signal peptide or the native intracellular and transmembrane domains of the homolog. Those skilled in the art will recognize other such additional sequences such as affinity tag sequences or other sequences that facilitate TLR activity, function or purification.

Such peptide chains may be readily made by determining the percent identity between a TLR3 homolog and SEQ ID NO: 6 as described above, selecting a homolog with at least 75% identity to SEQ ID NO: 6, identifying a position aligning to N221, N226, N387, or N636 of SEQ ID NO: 6, and introducing at least one mutation within 3 residues of the position identified. Importantly, the core consensus glycosylation site motif is the 3 amino acid residue Asn-X-Ser/Thr motif. Such mutations may be substitutions, deletions, or insertions and can be generated using in vitro and in vivo mutagenesis techniques well know in the art. Such peptide chains may also comprise additional mutations incorporated into the TLR3 homolog that do not occur within 3 amino acid residues of a position aligning to N221, N226, N387, or N636 of the amino acid sequence SEQ ID NO: 6.

Another aspect of the invention is a peptide chain comprising at least one mutation within 3 amino acid residues of position N221, N226, N387, or N636 of the amino acid sequence SEQ ID NO: 6. The amino acid sequences of SEQ ID NO: 8, SEQ ID NO: 10, SEQ ID NO: 12, SEQ ID NO: 16, SEQ ID NO: 18 and SEQ ID NO: 20 are exemplary of such peptide chains. Such mutations may be substitutions, deletions, or insertions and can be generated using in vitro and in vivo mutagenesis techniques well know in the art. Such peptide chains may also comprise additional sequences as discussed above.

Another aspect of the invention is a peptide chain comprising the amino acid sequence SEQ ID NO: 8, SEQ ID NO: 10, SEQ ID NO: 12, SEQ ID NO: 14, SEQ ID NO: 16, SEQ ID NO: 18, or SEQ ID NO: 20. Such peptide chains may also comprise additional sequences as discussed above.

In one embodiment the invention provides a nucleic acid encoding a peptide chain of the invention.

In another embodiment the invention provides a nucleic acid comprising the nucleic acid sequence of SEQ ID NO: 7, SEQ ID NO: 9, SEQ ID NO: 11, SEQ ID NO: 13, SEQ ID NO: 15, SEQ ID NO: 17, or SEQ ID NO: 19.

The nucleic acids of the invention may be made using in vivo or in vitro techniques known by those skilled in the art. Such nucleic acids may comprise DNA and RNA. Additionally, such nucleic acids may comprise additional nucleic acid sequences or be conjugated to another class of molecules. For example, such nucleic acids may be inserted into vector nucleic acids or supplied to a cell or system to produce expression of a peptide chain encoded by the nucleic acid. Such cells or systems may be eukaryotic cells, prokaryotic cells, archael cells, or cell free in vitro systems such as in vitro coupled transcription and translation systems. Techniques for peptide chain expression, introducing nucleic acids into vectors and supplying nucleic acids to cells, and cells or systems suitable for expression of peptide chains encoded by a nucleic acid are well know to those skilled in the art.

Another aspect of the invention is a method of modulating TLR3 activity in a cell comprising decreasing TLR3 glycosylation in the cell using techniques such as gene inactivation or transcript targeting (e.g. siRNA). TLR3 glycosylation may decreased, for example, by providing a cell with small molecules that inhibit TLR3 glycosylation such as tunicamycin, providing TLR3 homologs in which a glycosylation site has been modified so that it can no longer be glycosylated, over expressing glycolytic enzymes in the cell, and decreasing or inactivating expression of glycoslyases in the cell. Antibody molecules or antibody fragments may also be used to decrease TLR3 glycosylation and activity. Such antibody molecules or fragments may block ligand or receptor multimerization by binding to the region of cell surface TLR3 containing residue N221, N226, N387 or N636 of SEQ ID NO: 6. In the methods of the invention both N-glycosylation and O-glycosylation of TLR3 homologs may be targeted to decrease TLR3 glycosylation.

One aspect of the invention is a method of modulating TLR3 activity in a cell comprising providing tunicamycin to the cell. Tunicamycin can be provided to a cell in vitro via the culture media (at a concentration of about 0.2 to 0.5 μg/ml or in vivo via intravenous injection for example. Those skilled in the art will recognize many other methods and routes by which tunicamycin can be provided to a cell in vitro or in vivo.

In another embodiment the invention provides a method of modulating TLR3 activity in a cell comprising providing a peptide chain of the invention to the cell. Peptide chains may be provided to a cell by, for example, supplying the peptide chain to the fluid or tissue surrounding a cell, introducing an exogenous nucleic acid expressing the peptide chain into the cell, microinjection of the peptide chain into the cell, or fusing a cell with a second cell or vesicle containing the peptide chain. Those skilled in the art will recognize other means by which to provide a peptide chain to a cell.

In another embodiment the invention provides a method of modulating TLR3 activity in a cell comprising providing a nucleic acid of the invention to the cell. The nucleic acids of the invention may be provided to a cell through the use of viral, plasmid, cellular, or vesicular vectors, microinjection, naturally occurring nucleic acid uptake or competency, conjugation to molecule capable of entering a cell or by any other technique known to those skilled in the art by which nucleic acids may be introduced into a cell.

In another embodiment of the invention the nucleic acid is provided to the cell by transfection. Transfection of nucleic acids into a cell may be accomplished by a variety of techniques such as electroporation, chemical shock, lipofection, vescicle fusion, and other techniques known by those skilled in the art.

The present invention will now be described with reference to the following specific, non-limiting examples.

### EXAMPLE 1

### Glycosylation of hTLR3 Extracellular Domain

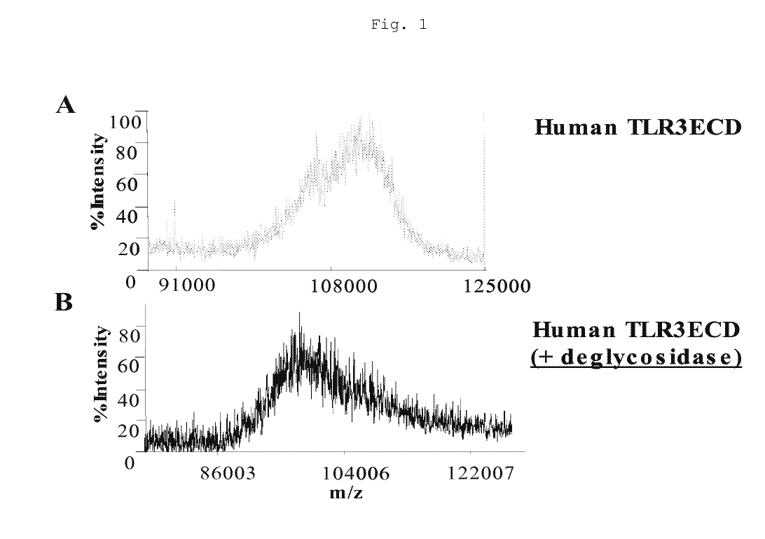

Mass spectroscopic analysis performed on the purified, soluble ECD of hTLR3 recombinantly expressed in Homo sapiens derived HEK293 cells (ATCC® number: CRL-1573™) revealed a high degree of charge heterogeneity among ionized hTLR3 ECD fragment species (FIG. 1A) and an average ion fragment mass of 110 kD. Treatment of recombinantly expressed hTLR3 ECD with a mixture of deglycosidases specific for O-linked and N-linked glycosylation followed by mass spectroscopic analysis revealed that deglycosidase treatment decreased the average ion fragment mass to 94 kD (FIG. 1B). Additionally, incubation of hTLR3 ECD with N-acetylneuraminic acid (NANAse), O-glycosidase DS, peptide N-Glycosidase F (PNGase F), or a cocktail containing all of these enzymes decreased glycoprotein specific staining of SDS-PAGE resolved hTLR3 ECD. Together these results indicate that the hTLR3 ECD is both N- and O-glycosylated.

hTLR3 ECD for mass spectroscopic and glycoprotein specific SDS-PAGE analyses were prepared as follows. First, a cDNA (SEQ ID NO: 1) encoding the full-length Homo sapiens TLR3 protein (SEQ ID NO: 2) and identical to accession number U88879 was cloned into pcDNA3.1. Next, a cDNA fragment encoding the hTLR3 ECD (SEQ ID NO: 3) and consisting of amino acids 1-703 of full length hTLR3 (SEQ ID NO: 4) was cloned into pcDNA3.1. This cDNA fragment was cloned into pcDNA3.1 in frame and on the 5′ side of a cDNA encoding a hexahistidine affinity tag. The resulting plasmid encodes a recombinant hTLR3 ECD comprising a C-terminal hexahistidine tag.

hTLR3 ECD encoded by this plasmid was expressed, processed, and secreted by transiently transfected HEK293 cells. Cells were transfected and cultured using standard methods. The recombinant hTLR3 ECD with carboxy terminal hexahistidine affinity tag was purified from cell culture supernatant using a Ni-NTA resin and was further purified by ion exchange chromatography using standard methods. The majority of hTLR3 ECD protein produced by this process is predicted to lack the first 26 amino terminal, signal peptide residues due to post-translational proteolytic processing and secretion. The hTLR3 ECD protein purified comprised amino acid residues 27 to 703 (SEQ ID NO: 6) of full length hTLR3 (SEQ ID NO: 2).

Mass spectroscopic analyses of purified, recombinant hTLR3 ECD were performed as follows. First, samples were concentrated with a Nanosep™ centrifugation concentrator (Pall Corp., East Hills, N.Y.), desalted using C-18 Zip Tips (Millipore Corp., Billerica, Mass.), and eluted with 50% acetonitrile/0.1% trifluoroacetic acid. 1 μL of each sample was then co-crystallized with the matrix in 2,5-dihydroxybenzoic acid in acetonitrile/water (50:50) containing 0.1% trifluoroacetic acid (TFA). MALDI-TOF experiments were then performed using standard methods and recorded using an ABI Voyager-DE™ STR mass spectrometer. To examine glycosylation, 1 μg of the purified hTLR3 ECD protein was incubated with a mixture of N-glycanase, O-glycanase, and sialidase at 37° C. for 24 h. Products of this incubation were then analyzed by MALDI-TOF spectroscopy as described above.

Preparation of purified hTLR3 ECD for SDS-PAGE resolution and glycoprotein specific visualization was performed as follows. First, samples containing recombinantly expressed hTLR3 ECD alone, hTLR3 ECD incubated with N-acetylneuraminic acid (NANAse), hTLR3 ECD incubated with O-glycosidase DS, peptide N-Glycosidase F (PNGase F), or a cocktail containing all of these enzymes and hTLR3 ECD were prepared. Samples containing equal amounts of hTLR3 ECD were then resolved by SDS-PAGE on a 4-12% gradient gel using standard methods and glycoprotein was visualized with the glycoprotein specific SYPRO Ruby protein gel stain (Invitrogen, Inc., Carlsbad, Calif.).

### EXAMPLE 2

### Effect of N-Glycosylation on Poly(I:C) Induced Activation of hTLR3 Signaling in Cells

In these experiments, TLR3 signaling was assayed using the pNF-κB-Luciferase (Stratagene, Inc., Carlsbad, Calif.) reporter gene construct transiently transfected by standard methods into HEK293 cells. This reporter construct comprised an NF-κB responsive DNA element linked to a luciferase reporter gene. Activation of TLR3 by poly(I:C) ligand increases NF-κB activity and results in activation of NF-κB responsive genes such as the luciferase reporter gene. HEK293 cells transfected with pNF-κB-Luciferase were transiently co-transfected with the control vector pHRL-TK constitutively producing a luciferase protein derived from Renilla. A cDNA encoding full-length hTLR3 was also transiently co-transfected, using standard methods, into HEK-293 cells transfected with the luciferase reporter gene.

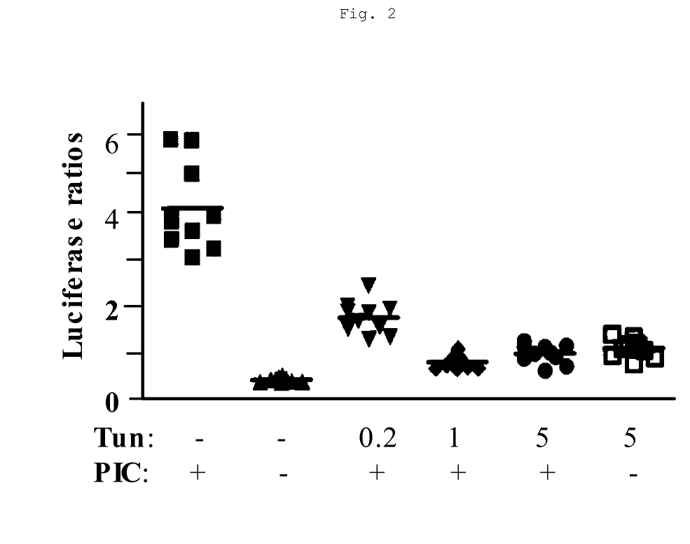

After transfection, cells were treated with non-toxic doses of tunicamycin as shown in FIG. 2. Tunicamycin inhibits N-linked glycosylation by inhibiting the attachment of N-acetylglucoasamine which is the first sugar residue attached to a peptide chain during glycosylation. Treated or control HEK293 cells were then incubated with 10 μg/ml of the hTLR3 ligand poly(I:C) (PIC) as indicated in FIG. 2.

After treatment, luciferase expressed from pNF-κB-Luciferase and pHRL-TK was assayed using standard methods. Data was expressed as a “luciferase ratio” equal to the pNF-κB-Luciferase activity normalized to pHRL-TK luciferase activity. Results are presented as scatter plots of data collected from 9 individual samples and is representative of 3 independently conducted, identical experiments.

### EXAMPLE 3

### Effect of hTLR3 ECD N-Glycosylation Inhibition on Poly(I:C) Induced Activation of hTLR3 Signaling in Cells

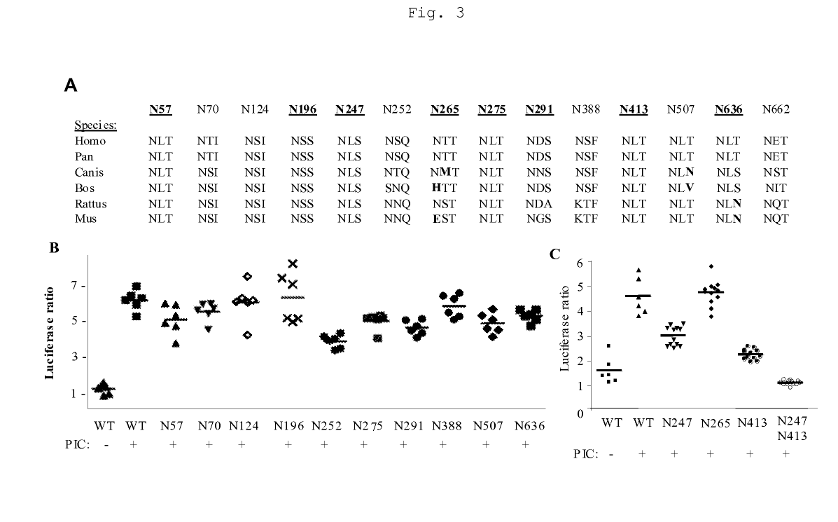

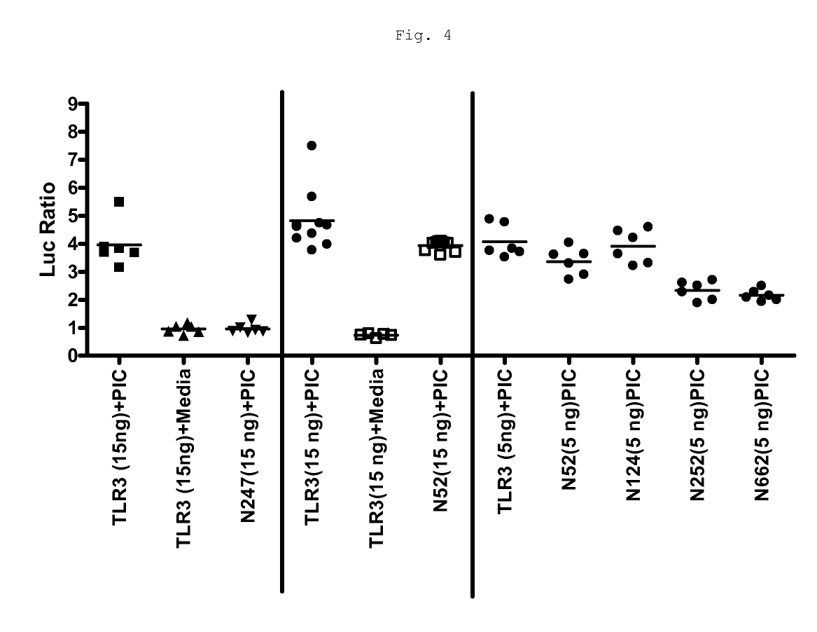

Surprisingly, two potential N-glycosylation sites, N247 and N413, of the many possible sites within the hTLR3 sequence of SEQ ID NO: 2 were found to play a critical role in hTLR3 signaling (FIG. 3C). Additionally, two other potential N-glycosylation sites in SEQ ID NO: 2, N252 and N662, were also found to play an important role in hTLR signaling (FIG. 3B and FIG. 4). Positions N247, N252, N413, and N662 in SEQ ID NO: 2 are respectively equivalent to N221, N226, N387, and N636 of SEQ ID NO: 6.

The hTLR3 ECD has several potential N-linked glycosylation sites, based on the presence of the N-glycosylation motif Asn-X-Ser/Thr (FIG. 3A) in various TLR3 homologs. Only five of these potential N-linked glycosylation sites were conserved between Homo sapiens, Pan troglodytes, Canis familiaris, Bos taurus, Rattus norvegicus and Mus musculus TLR3 homologs (FIG. 3A) as assessed by standard CLUSTALW alignment. The five conserved sites in SEQ ID NO: 2 were N57, N196, N247, N275, and N413. Four other potential N-glycosylation sites in SEQ ID NO: 2 varied between the various TLR3 homologs examined (FIG. 3A). These four other potential, albeit non-conserved, N-glycosylation sites were N252, N265, N291, N507, N636, and N662 of SEQ ID NO: 2.

The asparagines residues within these potential N-linked glycosylation sites in the hTLR3 ECD were individually mutated using standard methods to alanine residues (FIG. 3 and FIG. 4). Four additional asparagine residues present in the hTLR3 ECD that do not contain a match to the Asn-X-Ser/Thr N-glycosylation motif in any TLR3 homolog were also mutated as a control. These residues were N70, N124, and N388 of full length hTLR3 (SEQ ID NO: 2). All mutagenesis was performed on cDNAs encoding full-length hTLR3 in order to produce full-length, processed, and secreted hTLR3 with a mutagenized ECD. Mutated positions in SEQ ID NO: 2 are indicated in the figures by identification of the position of the alanine substitution (e.g. N70 means N70 has been replaced with an alanine residue).

Plasmids encoding and expressing the mutant hTLR3 cDNA constructs described above were individually transfected into HEK293T cells. Cells transfected with plasmids encoding individual, mutant hTLR3 molecules were also simultaneously transfected with the pNF-κB-Luciferase reporter and pHRL-TK luciferase control vector. Transfections and cell culture were performed using standard methods.

Transfected and control HEK293 cells were then incubated with 10 μg/ml (FIG. 3B and FIG. 4) or 2.5 μg/ml (FIG. 3C) of the hTLR3 ligand poly(I:C) (PIC) as indicated to assess the effect of the various hTLR3 mutations on hTLR3 signaling. After treatment luciferase expressed from pNF-κB-Luciferase and pHRL-TK was assayed using standard methods. Data was expressed as a “luciferase ratio” as described above. Results are presented as scatter plots of data collected from 6 individual samples.

The present invention now being fully described, it will be apparent to one of ordinary skill in the art that many changes and modifications can be made thereto without departing from the spirit or scope of the appended claims.

1. A peptide chain comprising an amino acid sequence with at least 75% identity to the amino acid sequence of SEQ ID NO: 6 and at least one mutation within 3 amino acid residues of a position aligning to N221, N226, N387, or N636 of the amino acid sequence SEQ ID NO: 6.

2. A peptide chain comprising at least one mutation within 3 amino acid residues of position N221, N226, N387, or N636 of the amino acid sequence SEQ ID NO: 6.

3. A peptide chain comprising the amino acid sequence SEQ ID NO: 8, SEQ ID NO: 10, SEQ ID NO: 12, SEQ ID NO: 14, SEQ ID NO: 16, SEQ ID NO: 18, or SEQ ID NO: 20.

4. A nucleic acid encoding the peptide chain of claim 1, 2 or 3.

5. The nucleic acid of claim 4 comprising the nucleic acid sequence of SEQ ID NO: 7, SEQ ID NO: 9, SEQ ID NO: 11, SEQ ID NO: 13, SEQ ID NO: 15, SEQ ID NO: 17, or SEQ ID NO: 19.

6. A method of modulating TLR3 activity in a cell comprising decreasing TLR3 glycosylation in the cell.

7. A method of modulating TLR3 activity in a cell comprising providing tunicamycin to the cell.

8. The method of claim 7 wherein the tunicamycin concentration outside the cell is about 0.2 μg/ml to 0.5 μg/ml.

9. A method of modulating TLR3 activity in a cell comprising providing the peptide chain of claim 1, 2, or 3 to the cell.

10. A method of modulating TLR3 activity in a cell comprising providing the nucleic acid of claim 4 to the cell.

11. The method of claim 10 wherein the nucleic acid is provided to the cell by transfection.
